(19) United States
(12) Patent Application Publication
(10) Pub. No.: US 2022/0403439 A1
(43) Pub. Date: Dec. 22, 2022

(54) Compositions for Replacing Chemical Surfactants

(71) Applicant: Locus IP Company, LLC, Solon, OH (US)

(72) Inventors: Ken ALIBEK, Solon, OH (US); Sean FARMER, Ft. Lauderdale, FL (US); Sergey KRAVTSOV, Solon, OH (US)

(21) Appl. No.: 17/769,890

(22) PCT Filed: Mar. 9, 2021
(86) PCT No.: PCT/US2021/021524

§ 371 Date: Apr. 18, 2022

Related U.S. Application Data
(60) Provisional application No. 62/987,529, filed on Mar. 10, 2020.

(51) Int. Cl. C12P 21/02; C12P 19/44; C09K 8/584; C07K 7/64; C07H 15/06

(52) U.S. Cl. CPC C12P 21/02; C12P 19/44; C09K 8/584; C07K 7/64; C07H 15/06

(57) ABSTRACT

The subject invention provides methods and compositions for replacing chemical surfactants for use in a wide variety of industrial applications. More specifically, the subject invention provides for the production of multi-functional biological surface-active compositions having one or more precise functional characteristics based on the desired use.

### CROSS-REFERENCE TO RELATED APPLICATION

This application claims priority to U.S. Provisional Patent Application No. 62/987,529, filed Mar. 10, 2020, which is incorporated herein by reference in its entirety.

### BACKGROUND OF THE INVENTION

Surfactants are surface-active, amphiphilic molecules with potential applications in nearly all areas of industry. Accordingly, the market for surfactants, which currently consists of thousands of different surface-active molecules, is growing rapidly. About 60% of surfactants are used as detergents and compounds for personal care products. Other uses include, for example, pharmaceuticals and supplements; oil and gas recovery; bioremediation; agriculture; cosmetics; coatings and paints; textile manufacture; food production and processing; and construction.

The properties of a surface-active molecule can be measured by hydrophile-lipophile balance (HLB). HLB is the balance of the size and strength of the hydrophilic and lipophilic moieties of a surface-active molecule. Specific HLB values are required to, for example, form a stable emulsion. In water/oil and oil/water emulsions, the polar moiety of the surface-active molecule orients towards the water, and the non-polar group orients towards the oil, thus lowering the interfacial tension between the oil and water phases.

HLB values range from 0 to about 20, with lower HLB (e.g., 10 or less) being more oil-soluble and suitable for water-in-oil emulsions, and higher HLB (e.g., 10 or more) being more water-soluble and suitable for oil-in-water emulsions. Other properties, such as foaming, wetting, detergency and solubilizing capabilities, are also dependent upon HLB.

Synthetic and chemical surfactants are advantageous because they can be easy to produce and can be tailored to perform a desired function based on their molecular structure. Thus, thousands of different surfactants have been developed, each having a certain narrow function. While this leaves ample options to choose from when producing products in which surfactants are used, the specificity of surfactant functions means that more varieties and combinations of surfactants are required for producing products with multiple functions. For example, a surfactant useful as a wetting agent may not necessarily be useful as a detergent, and a surfactant useful as an emulsifier may not necessarily be useful as an anti-corrosion agent.

The result is the over-use and over-production of chemical surfactants over the course of many decades. With growing consumer and regulatory awareness, the shortcomings of chemical surfactants are beginning to surface, including, for example, their narrow activity; potential and known toxicity to humans and animals; persistence in the environment, including aquatic environments, soil and ground water; contribution to climate change during production and application; and incompatibility with other chemicals.

Attempts have been made to create biologically-based surface-active molecules that are biodegradable and have low toxicity, but they are more difficult to modify in order to produce products having specific physical and chemical characteristics. One specific group of biological surface-active molecules includes those produced by microorganisms, or biosurfactants. Biosurfactants are a structurally diverse group of surface-active substances consisting of two parts: a polar (hydrophilic) moiety and non-polar (hydrophobic) group.

Due to their amphiphilic structure, biosurfactants can, for example, increase the surface area of hydrophobic water-insoluble substances, increase the water bioavailability of such substances, and change the properties of bacterial cell surfaces. Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase. The ability of biosurfactants to form pores and destabilize biological membranes also permits their use as antibacterial, antifungal, and hemolytic agents to, for example, control pests and/or microbial growth.

Typically, the hydrophilic group of a biosurfactant is a sugar (e.g., a mono-, di-, or polysaccharide) or a peptide, while the hydrophobic group is typically a fatty acid. Thus, there are countless potential variations of biosurfactant molecules based on, for example, type of sugar, number of sugars, size of peptides, which amino acids are present in the peptides, fatty acid length, saturation of fatty acids, additional acetylation, additional functional groups, esterification, polarity and charge of the molecule.

These variations lead to a group of molecules comprising a wide variety of classes, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. Each type of biosurfactant within each class can further comprise subtypes having further modified structures.

Like chemical surfactants, each biosurfactant molecule has its own HLB value depending on its structure; however, unlike production of chemical surfactants, which results in a single molecule with a single HLB value or range, one cycle of biosurfactant production typically results in a mixture of biosurfactant molecules (e.g., subtypes and isomers thereof), each of which has its own HLB. Thus, biosurfactant mixtures collected from a single microbial culture typically have varying, imprecise HLB values due to the variability of the biological processes involved in producing them.

Surfactants are a crucial aspect of industrial productivity across the globe. Because of growing challenges to the surfactant industry, including, for example, growing awareness of toxicity and pollution caused by certain surfactants; environmental and health regulations; and a societal trend towards a desire for “green” products, improved approaches to producing and using surface-active molecules are needed.

### BRIEF SUMMARY OF THE INVENTION

The subject invention provides methods and compositions for replacing chemical surfactants for use in a wide variety of industrial applications. More specifically, the subject invention provides for the production of multi-functional biological surface-active compositions having one or more precise functional characteristics based on the desired use. Advantageously, these compositions are, in preferred embodiments, non-toxic, biodegradable, and environmentally-friendly to produce and use.

In certain embodiments, customizable biosurfactant compositions are provided, comprising one or more biosurfactant molecules, wherein the identity, ratio and/or molecular structure of the one or more biosurfactants is pre-determined in order to achieve specific functional properties for the composition based on the desired use(s) for the composition.

In certain specific embodiments, a green surfactant composition having one or more desired functional properties is provided, the composition comprising one or more biosurfactant molecules, wherein the identity, ratio and structure of the one or more biosurfactant molecules are chosen based on their contribution to the desired functional properties.

In some embodiments, the functional properties are measured by, e.g., hydrophile-lipophile balance (HLB), critical micelle concentration (CMC), and/or kauri-butanol value (KB).

In certain embodiments, the composition comprises one or more biosurfactant molecules belonging to classes selected from, for example, glycolipids, lipopeptides, flavolipids, phospholipids, fatty acid ester compounds, lipoproteins, lipopolysaccharide-protein complexes, and polysaccharide-protein-fatty acid complexes.

In some embodiments, the composition comprises multiple biosurfactant molecules belonging to the same biosurfactant class. In some embodiments, the composition comprises biosurfactant molecules belonging to more than one of these biosurfactant classes.

In some embodiments, the composition comprises a glycolipid, such as, for example, a sophorolipid, rhamnolipid, trehalose lipid, cellobiose lipid and/or mannosylerythritol lipid. In some embodiments, the composition comprises a lipopeptide, such as, for example, a surfactin, fengycin, arthrofactin, lichenysin, iturin and/or viscosin.

Advantageously, including multiple biosurfactant molecules in the composition at certain pre-determined ratios creates a composition with broader ranges of either hydrophilicity or hydrophobicity. Additionally, the composition can be useful for multiple functions concurrently, even functions requiring, e.g., different HLB values or HLB ranges. In other words, one biological product comprising one or more biosurfactant molecules can replace a wide range of chemical products in an environmentally-friendly manner (see FIG. 1).

In additional and/or alternative embodiments, the composition can be tailored to have a specific, and in some instances, very precise, HLB value based on the identity and ratio of biosurfactant molecules within the composition.

In certain embodiments, the compositions can be used to replace compositions comprising chemical surfactants such as, for example, alkyl benzene sulfonates, linear alkyl benzene sulfonates, alcohol ethoxylates, diethanolamine, triethanolamine, alkyl ammonium chloride, alkyl glucosides, and others described herein.

In preferred embodiments, the subject invention provides methods for producing a “green” surfactant composition having one or more desired functional properties, the methods comprising identifying a biosurfactant molecule having a specific functional property and producing the biosurfactant molecule by cultivating a biosurfactant-producing microorganism under conditions favorable for production of the biosurfactant.

In certain embodiments, the method further comprises combining the biosurfactant molecule with one or more additional biosurfactant molecules, the identity, ratio and/or molecular structure of which are determined based on the desired use(s) for the composition. Thus, a composition is produced having one or more desired functional characteristics, including, for example, surface/interfacial tension reduction, viscosity reduction, emulsification, demulsification, solvency, detergency, and/or anti-microbial action.

In some embodiments, the method comprises modifying the structure of a biosurfactant molecule prior to using it in the composition.

In some embodiments, the identity, ratio and/or molecular structure of biosurfactant molecules in the green surfactant composition is determined based on, e.g., HLB, CMC, and/or KB, of the individual molecules. In some embodiments, the identity, ratio and/or molecular structure of biosurfactant molecules is determined based on a theoretical or actual desired HLB, CMC, and/or KB value for the composition as a whole.

In preferred embodiments, the green surfactant composition can be utilized in place of chemical surfactant(s) in products that would typically comprise the chemical surfactant(s), where one or more biosurfactants are chosen that have the same or similar functional properties as the chemical surfactant(s).

Thus, in some embodiments, the methods comprise selecting a known composition comprising one or more chemical surfactants and, optionally, one or more additional components, and producing an environmentally-friendly version of the known composition by using a green surfactant composition of the subject invention in place of the chemical surfactant(s). The green surfactant composition can be mixed with the optional additional components if present.

In some embodiments, the methods and compositions of the subject invention perform better than methods and compositions utilizing competitive chemical surfactants. For example, in some embodiments, the structure and/or size of a biosurfactant utilized according to the subject invention allows for enhanced surface tension reduction and/or interfacial tension reduction over that achieved by a chemical surfactant. Advantageously, in certain embodiments, a lower dosage of a biosurfactant molecule according to the subject invention is required to achieve a desired reduction in surface tension and/or interfacial tension than is required of a competitive chemical surfactant.

Advantageously, the methods and compositions of the subject invention reduce the cost and environmental impacts typically caused by production and use of surfactants by reducing and/or replacing the need for chemical surfactants altogether.

### BRIEF DESCRIPTION OF THE FIGURES

FIG. 1 shows HLB values of certain chemical surfactants (top) and of SLP molecules (bottom). One SLP composition (denoted by the double-sided arrow marked with a black star) produced according to embodiments of the subject methods can replace multiple individual chemical surfactants.

FIG. 2 shows how modification of a SLP molecule can adjust the HLB value of the molecule.

FIG. 3 shows how modification of a lipopeptide molecule can adjust the HLB value of the molecule.

FIG. 4 shows a chart of applications for surface-active molecules and corresponding HLB values required for the applications. The chart also denotes whether more LSL or ASL is required in the composition in order to achieve such HLB ranges.

FIG. 5 shows how modification of a RLP molecule can adjust the HLB value of the molecule.

FIG. 6 shows a list of possible modified forms of rhamnolipid molecules, having different numbers of sugar moieties and/or fatty acids, different fatty acid lengths, and different degree of saturation in the fatty acids. Each of these 58 types has different characteristics, including HLB.

FIG. 7 shows how modification of a MEL molecule can adjust the HLB value of the molecule.

### DETAILED DESCRIPTION OF THE INVENTION

The present invention provides materials and methods for producing “green” surfactant compositions that can be used in the oil and gas industry, agriculture, cosmetics, health care and environmental cleanup, as well as for a variety of other applications. Specifically, the subject invention provides materials and methods for the production of universally-applicable biosurfactant-based compositions comprising one or more biosurfactant molecules, wherein the composition can be modified to exhibit one or more precise functional characteristics based on the types and ratios of the biosurfactant molecules therein.

Advantageously, the green surfactant compositions produced according to the subject methods can comprise a precise, pre-determined ratio of biosurfactant molecules, to obtain a specific functional product having, for example, a desired HLB, CMC and/or KB value, or a desired range of these values.

### Selected Definitions

As used herein, a “green” compound or material means at least 95% derived from natural, biological and/or renewable sources, such as plants, animals, minerals and/or microorganisms, and furthermore, the compound or material is biodegradable. Additionally, “green” compounds or materials are minimally toxic to humans and have a LD50>5000 mg/kg. A “green” product preferably does not contain any of the following: non-plant based ethoxylated surfactants, linear alkylbenzene sulfonates (LAS), ether sulfates surfactants or nonylphenol ethoxylate (NPE).

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹², or more CFU per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell.

The phrases “biosurfactant” and “biosurfactant molecule” include all forms, analogs, orthologs, isomers, and natural and/or anthropogenic modifications of any biosurfactant class (e.g., glycolipid) and/or subtype thereof (e.g., sophorolipid).

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Green Surfactant Compositions and Methods of Production

The subject invention provides green surfactant compositions and methods of producing and using these compositions as replacements for chemical surfactant compositions. More specifically, the subject invention provides for the production of universally-applicable biosurfactant-based compositions comprising one or more biosurfactant molecules, wherein the composition can be modified to exhibit one or more functional characteristics based on the desired use(s) by altering the identity, ratio and/or molecular structure of the biosurfactant molecules. In some embodiments, the functional properties are measured by, e.g., hydrophile-lipophile balance (HLB), critical micelle concentration (CMC), and/or kauri-butanol value (KB).

In preferred embodiments, the subject invention provides methods for producing a “green” surfactant composition having one or more desired functional properties, the methods comprising identifying a biosurfactant molecule having a specific functional property and producing the biosurfactant molecule by cultivating a biosurfactant-producing microorganism under conditions favorable for production of the biosurfactant.

In certain embodiments, the method further comprises combining the biosurfactant molecule with one or more additional biosurfactant molecules, the identity, ratio and/or molecular structure of which are determined based on the desired use(s) for the composition. Thus, a composition is produced having one or more desired functional characteristics, including, for example, surface/interfacial tension reduction, viscosity reduction, emulsification, demulsification, solvency, detergency, and/or anti-microbial action.

In some embodiments, the identity, ratio and/or molecular structure of biosurfactant molecules in the green surfactant composition is determined based on, e.g., HLB, CMC, and/or KB, of the individual molecules. In some embodiments, the identity, ratio and/or molecular structure of biosurfactant molecules is determined based on a theoretical or actual desired HLB, CMC, and/or KB value for the composition as a whole.

The one or more biosurfactants can be produced using small to large scale cultivation methods. Most notably, the methods can be scaled to an industrial scale, i.e., a scale that is suitable for use in supplying biosurfactants in amounts to meet the demand for commercial applications, for example, production of compositions for enhanced oil recovery. In preferred embodiments, the biosurfactants are produced, optionally modified, and mixed at a centralized location that is, in some embodiments, not more than 300 miles, 200 miles, 100 miles, or 10 miles from where the green surfactant composition will be used.

The microorganisms utilized for producing the biosurfactants may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Mortierella, Mycorrhiza, Meyerozyma guilliermondii, Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mojavensis, B. mucilaginosus, B. subtilis), Burkholderia (e.g., B. thailandensis), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Paenibacillus polymyxa, Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the additional microorganisms are Bacillus spp. bacteria. In one specific embodiment, the Bacillus sp. is B. subtilis strain B1, B2 or B3 (see U.S. Pat. No. 10,576,519, which is incorporated by reference in its entirety), or B. subtilis subp. locus B4. In a specific embodiment, the Bacillus is B. amyloliquefaciens strain NRRL B-67928 (“B. amy”).

A culture of the B. amyloliquefaciens “B. amy” microbe has been deposited with the Agricultural Research Service Northern Regional Research Laboratory (NRRL), 1400 Independence Ave., S.W., Washington, D.C., 20250, USA. The deposit has been assigned accession number NRRL B-67928 by the depository and was deposited on Feb. 26, 2020.

The subject culture has been deposited under conditions that assure that access to the culture will be available during the pendency of this patent application to one determined by the Commissioner of Patents and Trademarks to be entitled thereto under 37 CFR 1.14 and 35 U.S.C. 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In one embodiment, the method comprises inoculating a fermentation reactor comprising a liquid growth medium with a biosurfactant-producing microorganism to produce a culture; and cultivating the culture under conditions favorable for production of the biosurfactant.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, biosurfactants concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

In certain embodiments, the cultivation method utilizes submerged fermentation in a liquid growth medium. In one embodiment, the liquid growth medium comprises a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more. In preferred embodiments, a hydrophilic carbon source, e.g., glucose, and a hydrophobic carbon source, e.g., oil or fatty acids, are used.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid. In certain embodiments, dissolved oxygen (DO) levels are maintained at about 25% to about 75%, about 30% to about 70%, about 35% to about 65%, about 40% to about 60%, or about 50% of air saturation.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the culture should be suitable for the microorganism of interest, and can be altered as desired in order to produce a specific biosurfactant molecule in the culture. Buffers, and pH regulators, such as carbonates and phosphates, may be used to stabilize pH near a preferred value.

In some embodiments, the pH is about 2.0 to about 7.0. In some embodiments, the pH is about 2.5 to about 5.5, about 3.0 to about 4.5, or about 3.5 to about 4.0. In one embodiment, the cultivation may be carried out continuously at a constant pH. In another embodiment, the cultivation may be subject to changing pH.

In one embodiment, the method of cultivation is carried out at about 5° to about 100° C., about 15° to about 60° C., about 20° to about 45° C., about 22° to about 30° C., or about 24° to about 28° C. In one embodiment, the cultivation may be carried out continuously at a constant temperature. In another embodiment, the cultivation may be subject to changing temperatures.

According to the subject methods, the microorganisms can be incubated in the fermentation system for a time period sufficient to achieve a desired effect, e.g., production of a desired amount of cell biomass or a desired amount of one or more microbial growth by-products. The microbial growth by-product(s) produced by microorganisms may be retained in the microorganisms and/or secreted into the growth medium. The biomass content may be, for example from 5 g/I to 180 g/l or more, or from 10 g/l to 150 g/l.

In certain embodiments, fermentation of the yeast culture occurs for about 48 to 150 hours, or about 72 to 150 hours, or about 96 to about 125 hours, or about 110 to about 120 hours. After the fermentation cycle is complete, the method can comprise, in some embodiments, extracting, concentrating and/or purifying the biosurfactant molecule.

In certain embodiments, the methods of the subject invention can be carried out in such a way that minimal-to-zero waste products are produced, thereby reducing the amount of fermentation waste being drained into sewage and wastewater systems, and/or being disposed of in landfills.

The cell biomass collected from the culture after extraction of the biosurfactant would typically be inactivated and disposed of. However, the subject methods can further comprise collecting the cell biomass and using it, in live or inactive form, for a variety of purposes, including but not limited to, as a soil amendment, a livestock feed supplement, an oil well treatment, and/or a skincare product. The cell biomass can be used directly, or it can be mixed with additives specific for the intended use.

In some embodiments, water or other non-toxic liquids used to extract and/or purify the biosurfactant can contain residual biosurfactants, nutrients and/or cell matter. Thus, in certain embodiments, the liquids can be used in irrigation drip lines or sprinklers as a soil or foliar treatment for plants; as a safe nutritional and/or hydration supplement for humans and animals; as a cleaning composition; and/or for countless other uses to reduce fermentation waste products.

In some embodiments, the method comprises modifying the structure of a biosurfactant molecule prior to adding it to the composition.

In some embodiments, adjusting the parameters of fermentation results in modification and/or production of one or more specific biosurfactant molecules in the culture, and/or production of a specific ratio of multiple biosurfactant molecules. These parameters can include, for example, using a specific strain of microorganism, adjusting the growth medium composition, co-cultivating the microbe with an antagonistic and/or influencing microbe, adding inhibitors and/or stimulant compounds to the nutrient medium, adjusting the temperature, pH and/or aeration of fermentation, and others.

In some embodiments, the biosurfactant molecule(s) obtained from the fermentation cycle can be modified post-fermentation by, for example, esterification, polymerization, addition of amino acids, addition of metals, and alteration of fatty acid chain lengths.

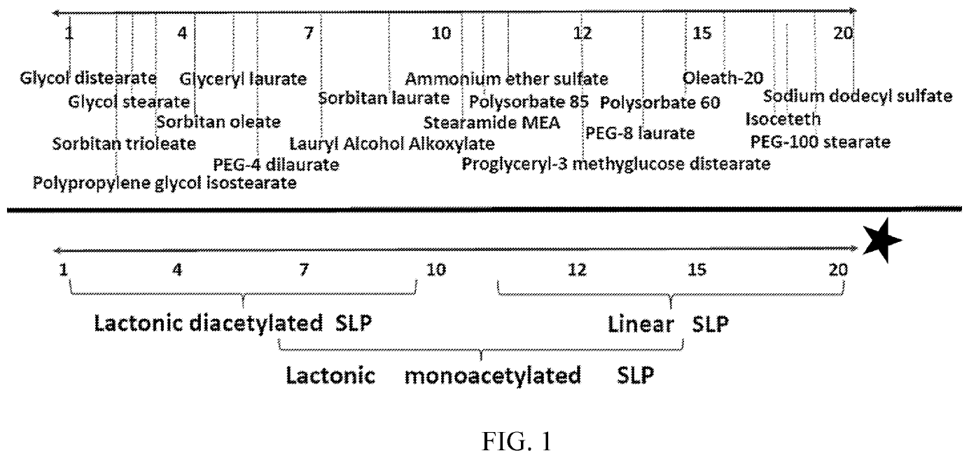

Advantageously, including multiple biosurfactant molecules in the composition at certain pre-determined ratios creates a composition with broader ranges of either hydrophilicity or hydrophobicity. Additionally, the composition can be useful for multiple functions concurrently, even functions requiring, e.g., different HLB values or HLB ranges. In other words, one biological product comprising one or more biosurfactant molecules can replace a wide range of chemical products in an environmentally-friendly manner (see FIG. 1).

In additional and/or alternative embodiments, the composition can be tailored to have a specific, and in some instances, very precise, HLB value based on the identity and ratio of biosurfactant molecules within the composition.

In certain embodiments, the composition comprises one or more biosurfactant molecules belonging to a class selected from, for example, glycolipids, lipopeptides, flavolipids, phospholipids, fatty acid ester compounds, lipoproteins, lipopolysaccharide-protein complexes, and polysaccharide-protein-fatty acid complexes.

In some embodiments, the composition comprises multiple biosurfactant molecules belonging to the same biosurfactant class. In some embodiments, the composition comprises biosurfactant molecules belonging to more than one of these biosurfactant classes.

In some embodiments, the composition comprises a glycolipid, such as, for example, a sophorolipid, rhamnolipid, trehalose lipid, cellobiose lipid and/or mannosylerythritol lipid.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a sophorolipid molecule. A “sophorolipid” or a “sophorolipid molecule” can include, for example, acidic (linear) (ASL) and lactonic (LSL) sophorolipids, and all possible derivatives thereof, including, for example, mono-acetylated sophorolipid, di-acetylated sophorolipid, esterified sophorolipid, sophorolipids with varying hydrophobic chain lengths, sophorolipid-metal complexes, sophorolipids with fatty acid-amino acid complexes attached, and others as described herein.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a rhamnolipid molecule. A “rhamnolipid” or a “rhamnolipid molecule” can include, for example, mono- and di-rhamnolipids, and all possible derivatives therein, as well as other forms as described herein.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a mannosylerythritol lipid molecule. A “mannosylerythritol lipid” or a “mannosylerythritol lipid molecule” can include, for example, tri-acylated, di-acylated, mono-acylated, tri-acetylated, di-acetylated, mono-acetylated and non-acetylated MEL, as well as stereoisomers and/or constitutional isomers thereof. In certain specific embodiments, the MEL are characterized as groups: MEL A (di-acetylated), MEL B (mono-acetylated at C4), MEL C (mono-acetylated at C6), MEL D (non-acetylated), tri-acetylated MEL A, tri-acetylated MEL B/C, as well as other forms as described herein.

In some embodiments, the composition comprises 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a lipopeptide, such as, for example, a surfactin, fengycin, arthrofactin, lichenysin, iturin and/or viscosin.

In some embodiments, two or more purified biosurfactant molecules are mixed with one another. In some embodiments, two or more unpurified, or crude form, biosurfactants are mixed with one another, wherein the crude form can comprise, for example, residual nutrient medium, microbial cells, and/or other microbial metabolites produced during fermentation. In some embodiments, a purified biosurfactant molecule can be mixed with a crude form biosurfactant.

In preferred embodiments, the green surfactant composition can be utilized in place of chemical surfactant(s) in products that would typically comprise the chemical surfactant(s), where one or more biosurfactants are chosen that have the same or similar functional properties as the chemical surfactant(s).

Thus, in some embodiments, the methods comprise selecting a known composition comprising one or more chemical surfactants and, optionally, one or more additional components, and producing an environmentally-friendly version of the known composition by using a green surfactant composition of the subject invention in place of the chemical surfactant(s). The green surfactant composition can be mixed with the one or more optional additional components, if present.

In certain embodiments, the compositions can be used to replace compositions comprising chemical surfactants. Typical chemical or synthetic surfactants (meaning, non-biological surfactants) comprise a hydrophobic group, which is usually a long hydrocarbon chain (C8-C18) that may or may not be branched, while the hydrophilic group is formed by moieties such as carboxylates, sulfates, sulfonates (anionic), alcohols, polyoxyethylenated chains (nonionic) and quaternary ammonium salts (cationic).

Non-biological surfactants that can be replaced in surfactant compositions utilizing the methods and compositions of the subject invention include, but are not limited to: anionic surfactants, ammonium lauryl sulfate, sodium lauryl sulfate (also called SDS, sodium dodecyl sulfate), alkyl-ether sulfates sodium laureth sulfate (also known as sodium lauryl ether sulfate (SLES)), sodium myreth sulfate; docusates, dioctyl sodium sulfosuccinate, perfluorooctanesulfonate (PFOS), perfluorobutanesulfonate, linear alkylbenzene sulfonates (LABs), alkyl-aryl ether phosphates, alkyl ether phosphate; carboxylates, alkyl carboxylates (soaps), sodium stearate, sodium lauroyl sarcosinate, carboxylate-based fluorosurfactants, perfluorononanoate, perfluorooctanoate; cationic surfactants, pH-dependent primary, secondary, or tertiary amines, octenidine dihydrochloride, permanently charged quaternary ammonium cations, alkyltrimethylammonium salts, cetyl trimethylammonium bromide (CTAB) (a.k.a. hexadecyl trimethyl ammonium bromide), cetyl trimethylammonium chloride (CTAC), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, dioctadecyldi-methylammonium bromide (DODAB); zwitterionic (amphoteric) surfactants, sultaines CHAPS (3-[(3-Cholamidopropyl)dimethylammonio]-1-propanesulfonate), cocamidopropyl hydroxysultaine, betaines, cocamidopropyl betaine, phosphatidylserine, phosphatidylethanolamine, phosphatidylcholine, sphingomyelins; nonionic surfactants, ethoxylate, long chain alcohols, fatty alcohols, cetyl alcohol, stearyl alcohol, cetostearyl alcohol, oleyl alcohol, polyoxyethylene glycol alkyl ethers (Brij): CH3-(CH2)10-16-(O—C2H4)1-25-OH (octaethylene glycol monododecyl ether, pentaethylene glycol monododecyl ether), polyoxypropylene glycol alkyl ethers: CH3-(CH₂)10-16-(O—C3H6)1-25-OH, glucoside alkyl ethers: CH3-(CH₂)10-16-(O-Glucoside)1-3-0H (decyl glucoside, lauryl glucoside, octyl glucoside), polyoxyethylene glycol octylphenol ethers: C8H17-(C6H4)-(O—C2H4)1-25-OH (Triton X-100), polyoxyethylene glycol alkylphenol ethers: C9H19-(C6H4)-(O—C2H4)1-25-OH (nonoxynol-9), glycerol alkyl esters (glyceryl laurate), polyoxyethylene glycol sorbitan alkyl esters (polysorbate), sorbitan alkyl esters (spans), cocamide MEA, cocamide DEA, dodecyldimethylamine oxide, copolymers of polyethylene glycol and polypropylene glycol (poloxamers), and polyethoxylated tallow amine (POEA).

Anionic surfactants contain anionic functional groups at their head, such as sulfate, sulfonate, phosphate, and carboxylates. Prominent alkyl sulfates include ammonium lauryl sulfate, sodium lauryl sulfate (also called SDS, sodium dodecyl sulfate) and the related alkyl-ether sulfates sodium laureth sulfate, also known as sodium lauryl ether sulfate (SLES), and sodium myreth sulfate. Carboxylates are the most common surfactants and comprise the alkyl carboxylates (soaps), such as sodium stearate.

Surfactants with cationic head groups include: pH-dependent primary, secondary, or tertiary amines; octenidine dihydrochloride; permanently charged quaternary ammonium cations such as alkyltrimethylammonium salts: cetyl trimethylammonium bromide (CTAB) a.k.a. hexadecyl trimethyl ammonium bromide, cetyl trimethylammonium chloride (CTAC); cetylpyridinium chloride (CPC); benzalkonium chloride (BAC); benzethonium chloride (BZT); 5-Bromo-5-nitro-1,3-dioxane; dimethyldioctadecylammonium chloride; cetrimonium bromide; and dioctadecyldi-methylammonium bromide (DODAB).

Zwitterionic (amphoteric) surfactants have both cationic and anionic centers attached to the same molecule. The cationic part is based on primary, secondary, or tertiary amines or quaternary ammonium cations. The anionic part can be more variable and include sulfonates. Zwitterionic surfactants commonly have a phosphate anion with an amine or ammonium, such as is found in the phospholipids phosphatidylserine, phosphatidylethanolamine, phosphatidylcholine, and sphingomyelins.

A surfactant with a non-charged hydrophilic part, e.g. ethoxylate, is non-ionic. Many long chain alcohols exhibit some surfactant properties.

### Compositions

In certain embodiments, multi-functional biosurfactant-based compositions are provided, comprising one or more biosurfactant molecules. In specific embodiments, the compositions can be customized for a specific purpose by adjusting identity, ratio and/or structure of the biosurfactant molecule(s).

In some embodiments, the identities, ratios and/or structures of the biosurfactant molecule(s) are adjusted to achieve a desired hydrophile-lipophile balance (HLB). In certain embodiments, each of the individual biosurfactant molecules in the composition acts upon the environment individually, such that, for example, a composition can be used for functions requiring high HLB and functions requiring low HLB. For example, a composition could be produced that is capable of oil-in-water emulsification (HLB 13-18) and water-in-oil emulsification (HLB 3-6). The strength of 0/W emulsification versus the strength of the W/O emulsification of the exemplary composition would depend upon the ratio of the high HLB molecule(s) to the low HLB molecule(s).

In certain additional and/or alternative embodiments, the biosurfactant-based composition is characterized by a specific, and in some instances, precise, HLB value as a whole, wherein the specific HLB value can be specially-tailored by adjusting the ratio of the biosurfactant molecule(s) in the composition.

In some embodiments, the identities, ratios and/or structures of the biosurfactant molecule(s) are adjusted to achieve a desired critical micelle concentration (CMC) value. CMC is the concentration of a surface active molecule or composition where aggregates of micelles form and all further surfactants that are added form micelles. Before reaching the CMC, the surface tension reduces as the concentration of the surfactant increases. After reaching the CMC, the surface tension is relatively constant.

In some embodiments, the identities, ratios and/or structures of the biosurfactant molecule(s) are adjusted to achieve a desired kauri-butanol (KB) value. KB is used for describing the solvent strength of a substance, as well as the substance's detergency power.

Further components can be added to the compositions as needed for a particular use. The additives can be, for example, buffers, carriers, other microbe-based compositions produced at the same or different facility, viscosity modifiers, preservatives, nutrients for microbe growth, nutrients for plant growth, solvents, pharmaceuticals, nutraceuticals, tracking agents, pesticides, herbicides, animal feed, disinfectants, builders, co-surfactants, fragrances, food ingredients and other ingredients specific for an intended use.

The present invention further provides uses for these products in many settings including, for example, improved bioremediation, mining, and oil and gas production; waste disposal and treatment; enhanced human health; enhanced health of livestock and other animals; food additives, such as preservatives and/or emulsifiers; cosmetic additives; and enhanced health and productivity of plants.

In some embodiments, the methods and compositions of the subject invention perform better than methods and compositions utilizing competitive chemical surfactants. For example, in some embodiments, the structure and/or size of a biosurfactant utilized according to the subject invention allows for enhanced surface tension reduction and/or interfacial tension reduction over that achieved by a chemical surfactant. Advantageously, in certain embodiments, a lower dosage of a biosurfactant molecule according to the subject invention is required to achieve a desired reduction in surface tension and/or interfacial tension than is required of a competitive chemical surfactant.

In certain embodiments, the size of a biosurfactant molecule and/or a micelle according to the subject invention is less than 10 nm, preferably less than 8 nm, more preferably less than 5 nm. In a specific embodiment, the size is from 0.8 nm to 1.5 nm, or about 1.0 to 1.2 nm. Advantageously, such small size allows for enhanced penetration of biosurfactants into nanometer-sized spaces and pores, such as those in subterranean oil-bearing formations, between plant and animal cells, in cell membranes, and in biofilm matrices.

Cultivation of microbial biosurfactants according to the prior art is a complex, time and resource consuming, process that requires multiple stages. Advantageously, the subject methods do not require complicated equipment or high energy consumption, and thus reduce the capital and labor costs of producing microorganisms and their metabolites on a large scale. Furthermore, only one product produced according to the subject invention is needed to perform a wide variety of surface-active functions, which can be used for any application where surfactants are used, for example, the oil and gas industry, agriculture industry, and/or cosmetics industry. Thus, the subject invention can be used to replace and/or reduce the usage of chemical surfactants in these industries.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Sophorolipid Production

Sophorolipids are glycolipid biosurfactants produced by, for example, various yeasts of the Starmerella clade. SLP consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-A9-octadecenoic acid. The hydroxy fatty acid is generally 16 or 18 carbon atoms, and may contain one or more unsaturated bonds. Furthermore, the sophorose residue can be acetylated on the 6- and/or 6′-position(s). The fatty acid carboxyl group can be free (acidic or linear form (General Formula 1)) or internally esterified at the 4″-position (lactonic form (General Formula 2)). S. bombicola produces a specific enzyme, called S. bombicola lactone esterase, which catalyzes the esterification of linear SLP to produce lactonic SLP.

In preferred embodiments, the SLP according to the subject invention are represented by General Formula (1) and/or General Formula (2), and are obtained as a collection of 30 or more types of structural homologues having different fatty acid chain lengths (R³), and, in some instances, having an acetylation or protonation at R¹ and/or R².

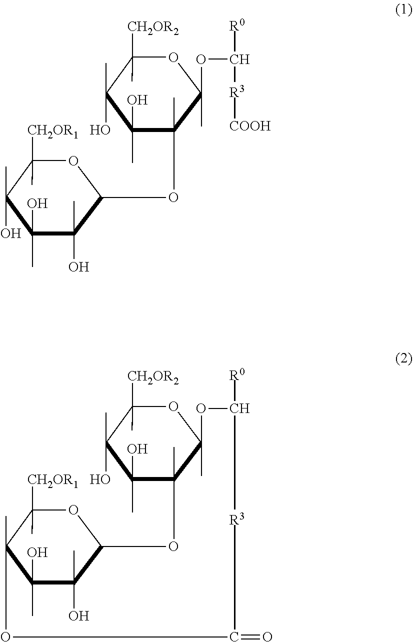

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Non-limiting examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and the like. R³ typically has 11 to 20 carbon atoms, preferably 13 to 17 carbon atoms, and more preferably 14 to 16 carbon atoms.

To produce SLP, a fermentation reactor is inoculated with Starmerella bombicola yeast. The temperature of fermentation is held at 23 to 28° C. After about 22 to 26 hours, the pH of the culture is set to about 3.0 to 4.0, or about 3.5, using 20% NaOH. The fermentation reactor comprises a computer that monitors the pH and controls the pump used to administer the base, so that the pH remains at 3.5.

After about 6-7 days of cultivation (120 hours+/−1 hour), if 7.5 ml of a SLP layer is visible with no oil visible and no glucose detected, the batch is ready for harvesting.

### Modifying SLP Products During Fermentation

The structure of the SLP molecules produced by the subject methods can be modified in multiple ways by altering fermentation parameters. One approach is to include long-chain fatty alcohols (e.g., C₄ to C₂₆-alcohols) in the nutrient medium. The resulting SLP molecules will comprise hydrophobic moieties up to C₃₆ in length, and will increase the hydrophobicity, emulsification and detergency capabilities of the composition.

Another approach is to limit the amount of sugar and/or oil in the fermentation medium. For example, in some embodiments, the amount of glucose is limited to about 25 g/L to about 75 g/L and/or the amount of canola oil is limited to about 25 ml/L to about 75 ml/L. In certain embodiments, this will increase the amount of ASL produced in the culture.

To increase the amount of hydrophobic SLP molecules (e.g., LSL and some ASL) the yeast is cultivated at a temperature of about 22° C. to about 28° C., and at a pH of about 2.5 to 4.0, where the pH begins at about 4.0 and reduces to—and is stabilized at—about 2.5 during cultivation.

To increase the amount of ASL in the culture, the yeast is cultivated at a pH of about 5.5, and at a temperature of about 35° C. Additionally, utilizing the yeast Candida kuoi can result in a composition comprising only ASL, as this yeast only produces ASL.

### Modifying SLP Products After Fermentation

Some modifications of SLP molecules occur after the cultivation cycle is ended. For example, inorganic acids, alkaline substances and/or salts can be mixed with SLP to alter solubility.

Furthermore, in addition to SLP, the yeasts also produce enzymes, such as lipases and esterases, into the yeast culture. Certain enzymes catalyze the bonding of amino acids to the SLP molecules. Thus, amino acids can be added to the yeast culture, and are chosen based on the character of the amino acid and the desired character of the SLP molecule(s). Cationic, anionic, polar and non-polar amino acids, when bonded to the SLP molecules, can alter the properties of the SLP molecules to be cationic, anionic, polar or non-polar.

Additionally, certain enzymes catalyze the esterification of the SLP molecules in the presence of the alcohol and fatty acid.

When the fermentation cycle is completed, an alcohol (e.g., 10% v/v) selected from methanol, ethanol, isopropyl alcohol, hexanol, or heptanol is added to the yeast culture. The liquid fermentation medium preferably already comprises a source of fatty acids, for example, canola oil. However, additional fatty acids can be added if a certain esterified product is desired, for example, purified forms of fatty acids such as palmitic, stearic, oleic, linoleic, linolenic, ricinoleic, lauric, and myristic acids.

The yeast culture with alcohol and fatty acid is mixed for 24 hours. After 24 hours, mixing is stopped and the culture will contain SLP esters containing an added alcohol, a sophorose, and a fatty acid ester, e.g., methanol sophorolipid oleic acid ester, which is formed when methanol and oleic acid are used.

### Example 2—Sample SLP Compositions

### Purified LSL

LSL produced and purified using a method according to an embodiment of the subject invention comprised 83.5% SLP (45.13% LSL and 38.36% ASL). Fatty acids (7.5%) and water (9%) comprised the remainder of the product. The HLB was between 1.65 and 2.99.

Despite the fact that ASL were present in the purified product, they were not treated as an impurity. ASL are generally hydrophilic by nature and LSL are generally lipophilic by nature. Here, the ASL exhibited lipophilic properties. Therefore, the properties of the composition were consistent with greater purity LSL, particularly in terms of HLB.

### Purified ASL

ASL produced and purified using a method according to an embodiment of the subject invention comprised 92% SLP (80% ASL and 12% LSL). Fatty acids (6%), glucose (2%) and water (0.5%) comprised the remainder of the product. The HLB was >20.

Here, the ASL exhibited the typical hydrophilic properties, while the small amount of lipophilic LSL is treated as an impurity that can be removed by further purification.

### Example 3—Adjustment of a Composition Comprising SLP for a Desired Function

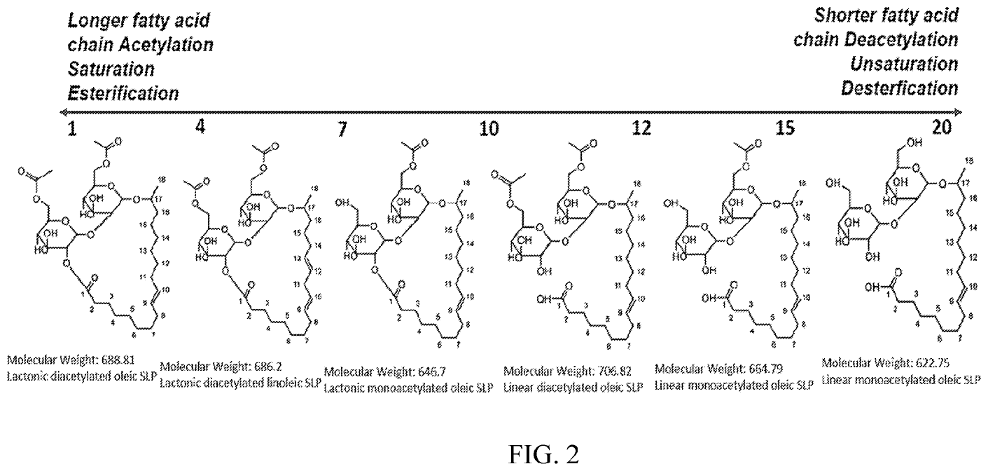

The following principles are referred to when adjusting the types and/or ratios of SLP molecules in the composition (Table 1; see also FIG. 2):

TABLE 1
Adjusting SLP composition to achieve desired function

| Desired functional | |
|---|---|
| characteristic | Composition Adjustment |
| Increased HLB | Increase ASL proportion |
| | Decrease fatty acid chain lengths and |
| | increase unsaturation of fatty acids |
| | Decrease esterification of SLP |
| | Decrease acetylation of SLP |
| Decreased HLB | Increase LSL proportion |
| | Increase fatty acid chain lengths and |
| | increase saturation of fatty acids |
| | Increase degree of esterification of |
| | esterified SLP |
| | Increase acetylation of SLP |
| Increased solubility | Increase ASL proportion |
| in water | |
| Increased foaming | Increase ASL proportion |
| Decreased viscosity | Increase LSL proportion |
| Increased anti- | Increase LSL proportion |
| microbial properties | |
| Increased CMC | Increase LSL proportion |
| Decreased CMC | Increase ASL proportion |
| | Increase esterified SLP proportion |
| | Utilize higher alcohols (>2 carbons) in |
| | esterification of SLP |
| Increased KB value | Utilize saturated fatty acids in |
| | esterification of SLP |
| | Decrease chain length of fatty acids used |
| | in esterification of SLP |
| Decreased KB value | Utilize higher alcohols in esterification |
| | of SLP |

### Example 4—Analysis of SLP Compositions

Fermentation of S. bombicola was repeated 50 times, resulting in production of 50 lots of SLP having the following ratios of LSL to ASL (Table 2):

TABLE 2
SLP composition for S. bombicola culture repetitions.

| SAMPLE | Percent of LSL | Percent of ASL | HLB |
|---|---|---|---|
| 1 | 70.51 | 29.48 | 11.44 |
| 2 | 70.61 | 29.39 | 4.32 |
| 3 | 71.66 | 28.34 | 4.32 |
| 4 | 70.44 | 29.56 | 12.86 |
| 5 | 76.04 | 23.96 | 12.33 |
| 8 | 80.01 | 19.98 | 9.66 |
| 9 | 69.98 | 30.01 | 12.33 |
| 10 | 75.85 | 24.15 | 9.66 |
| 11 | 61.17 | 38.83 | 12.33 |
| 12 | 68.18 | 31.82 | 11.44 |
| 13 | 64.56 | 35.44 | 12.86 |
| 14 | 84.77 | 15.23 | 4.32 |
| 15 | 84.43 | 15.56 | 9.66 |
| 16 | 82.79 | 17.2 | 4.32 |
| 17 | 53.22 | 46.78 | 22 |
| 18 | 97.89 | 2.1 | 1.65 |

Linear regression analysis was performed using the percentage of ASL in the lots. The following equation was obtained:

0.17+(0.365x % of ASL)=HLB value

This equation can be used to predict HLB value using the percentage of ASL in the SLP. Different ratios of LSL and ASL were mixed to see how the actual data would fit the hypothetical curve. The results support the calculated formula, as shown below (Table 3):

TABLE 3
HLB value based on percentage of ASL.

| Percent of acidic SLP | HLB |
|---|---|
| 0-5 | 1-3 |
| 5-16 | 3-6 |
| 16-25 | 6-9 |
| 26-35 | 9-12 |
| 36-45 | 12-16 |
| 46-55 | 18-22 |

### Example 5—Adjustment of a Composition Comprising RLP for a Desired Function

In some embodiments, the composition comprises a rhamnolipid (RLP). Rhamnolipids comprise a glycosyl head group (i.e., a rhamnose) moiety, and a 3-(hydroxyalkanoyloxy)alkanoic acid (HAA) fatty acid tail, such as, e.g., 3-hydroxydecanoic acid. Two main subtypes of rhamnolipids exist, mono- and di-rhamnolipids, which comprise one or two rhamnose moieties, respectively. The HAA moiety can vary in length and degree of branching, depending on, for example, the growth medium and the environmental conditions.

Rhamnolipids according to the subject invention can have the following structure:

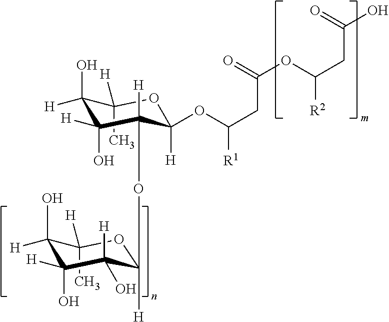

wherein m is 2, 1 or 0,

n is 1 or 0,

R¹ and R² are, independently of one another, the same or a different organic functional group having 2 to 24, preferably 5 to 13 carbon atoms, in particular a substituted or unsubstituted, branched or unbranched alkyl functional group, which can also be unsaturated,

wherein the alkyl functional group is a linear saturated alkyl functional group having 8 to 12 carbon atoms, or is a nonyl or a decyl functional group or a mixture thereof.

Salts of these compounds are also included according to the invention. In the present invention, the term “di-rhamnolipid” is understood to mean compounds of the above formula or the salts thereof in which n is 1. Accordingly, “mono-rhamnolipid” is understood in the present invention to mean compounds of the general formula or the salts thereof in which n is 0.

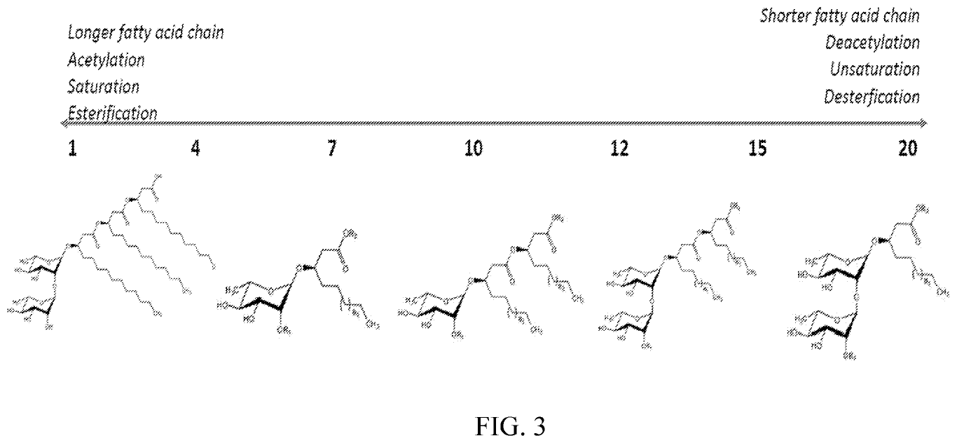

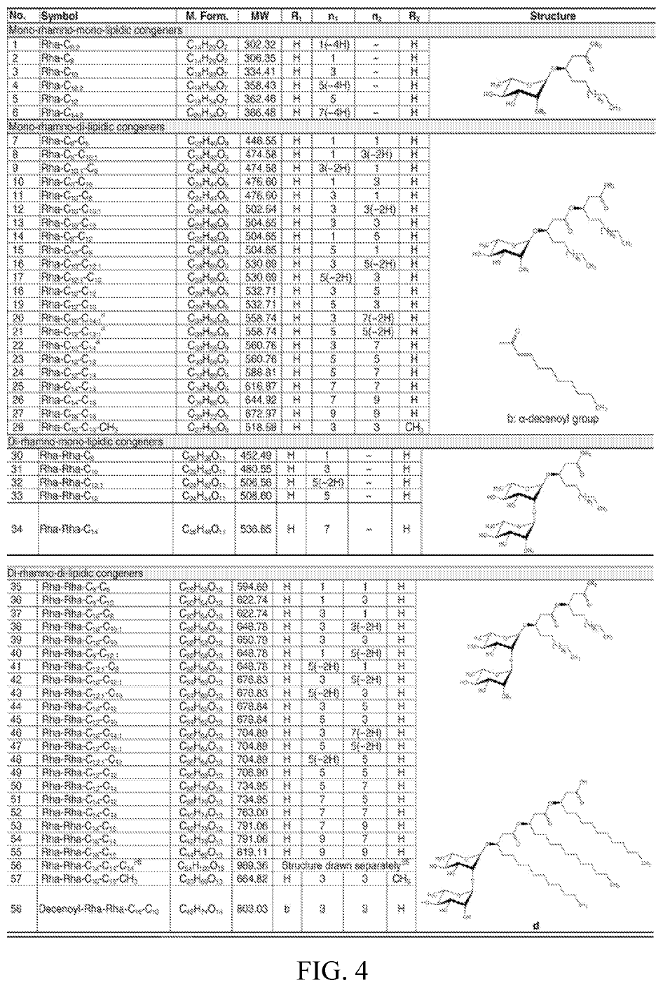

As shown in FIGS. 3-4, the structure of a RLP molecule can affect the function significantly.

### Example 6—Adjustment of a Composition Comprising MEL for a Desired Function

In some embodiments, the composition comprises a mannosylerythritol lipid (MEL), a class of biosurfactant comprising either 4-O—B-D-mannopyranosyl-meso-erythritol or 1-O—B-D-mannopyranosyl-meso-erythritol as the hydrophilic moiety, and fatty acid groups and/or acetyl groups as the hydrophobic moiety.

MEL subtypes can comprise different carbon-length chains or different numbers of acetyl and/or fatty acid groups. MEL subtypes can include, for example, tri-acylated, di-acylated, mono-acylated, tri-acetylated, di-acetylated, mono-acetylated and non-acetylated MEL, as well as stereoisomers and/or constitutional isomers thereof. Furthermore, there can be one to three esterified fatty acids, from 6 to 12 carbons, or more, in chain length.

In certain specific embodiments, the MEL are characterized as groups: MEL A (di-acetylated), MEL B (mono-acetylated at C4), MEL C (mono-acetylated at C6), MEL D (non-acetylated), tri-acetylated MEL A, tri-acetylated MEL B/C, and further including all possible isomers of the members of these groups. MEL according to the subject invention can have the following structure:

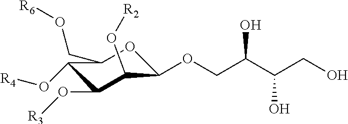

wherein R₂ and R₃═C₂-C₁₈ fatty acid, and
MEL A: R₄=R₆=acetyl group;
MEL B: R₄=H, R₆=acetyl group;
MEL C: R₄=acetyl group, R₆=H; and
MEL D: R₄=R₆=H

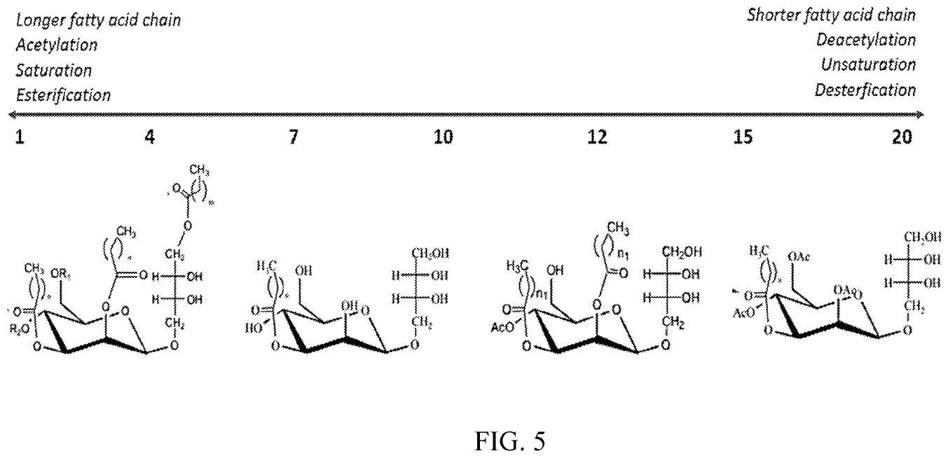

As shown in FIG. 5, the structure of a MEL molecule can affect the function significantly.

### Example 7—Adjustment of a Composition Comprising a Lipopeptide for A Desired Function

In some embodiments, the composition comprises a lipopeptide. Lipopeptides are oligopeptides synthesized by bacteria using large multi-enzyme complexes. They are frequently used as antibiotic compounds, and exhibit a wide antimicrobial spectrum of action, in addition to surfactant activities. All lipopeptides share a common cyclic structure consisting of a β-amino or β-hydroxy fatty acid integrated into a peptide moiety.

Surfactin lipopeptides consist of heptapeptides containing a β-hydroxy fatty acid with 13 to 15 carbon atoms. Fengycin lipopeptides, which include plipastatins, are decapeptides with a β-hydroxy fatty acid. Iturin lipopeptides, represented by, e.g., iturin A, mycosubtilin, and bacillomycin, are heptapeptides with a β-amino fatty acid.

Other lipopeptides have been identified, which exhibit a variety of useful characteristics. These include, but are not limited to, kurstakins, arthrofactin, viscosin, glomosporin, amphisin, and syringomycin, to name a few.

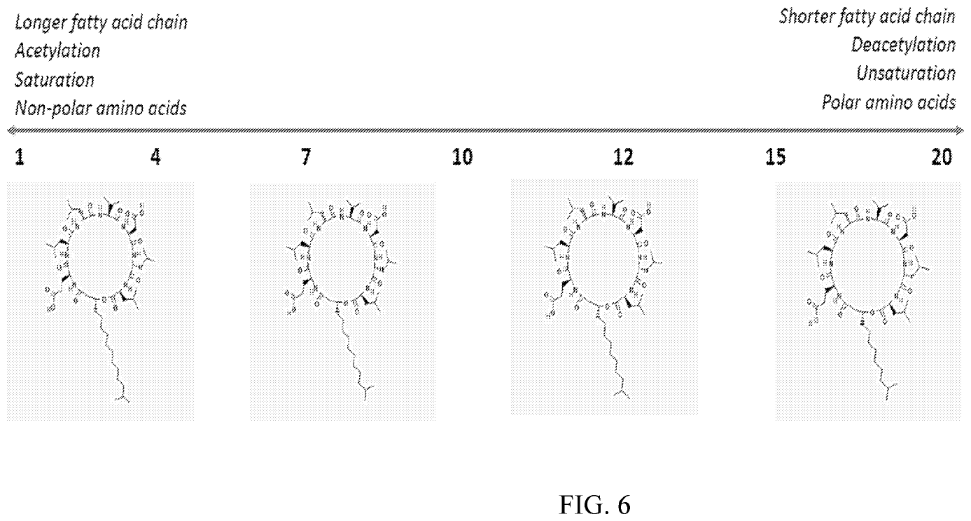

As shown in FIG. 6, the structure of a lipopeptide molecule can affect the function significantly.

In certain embodiments, the lipopeptide has one of the following general structures, where General Structure A is an iturin, General Structure B is a surfactin and General Structure C is a fengycin.

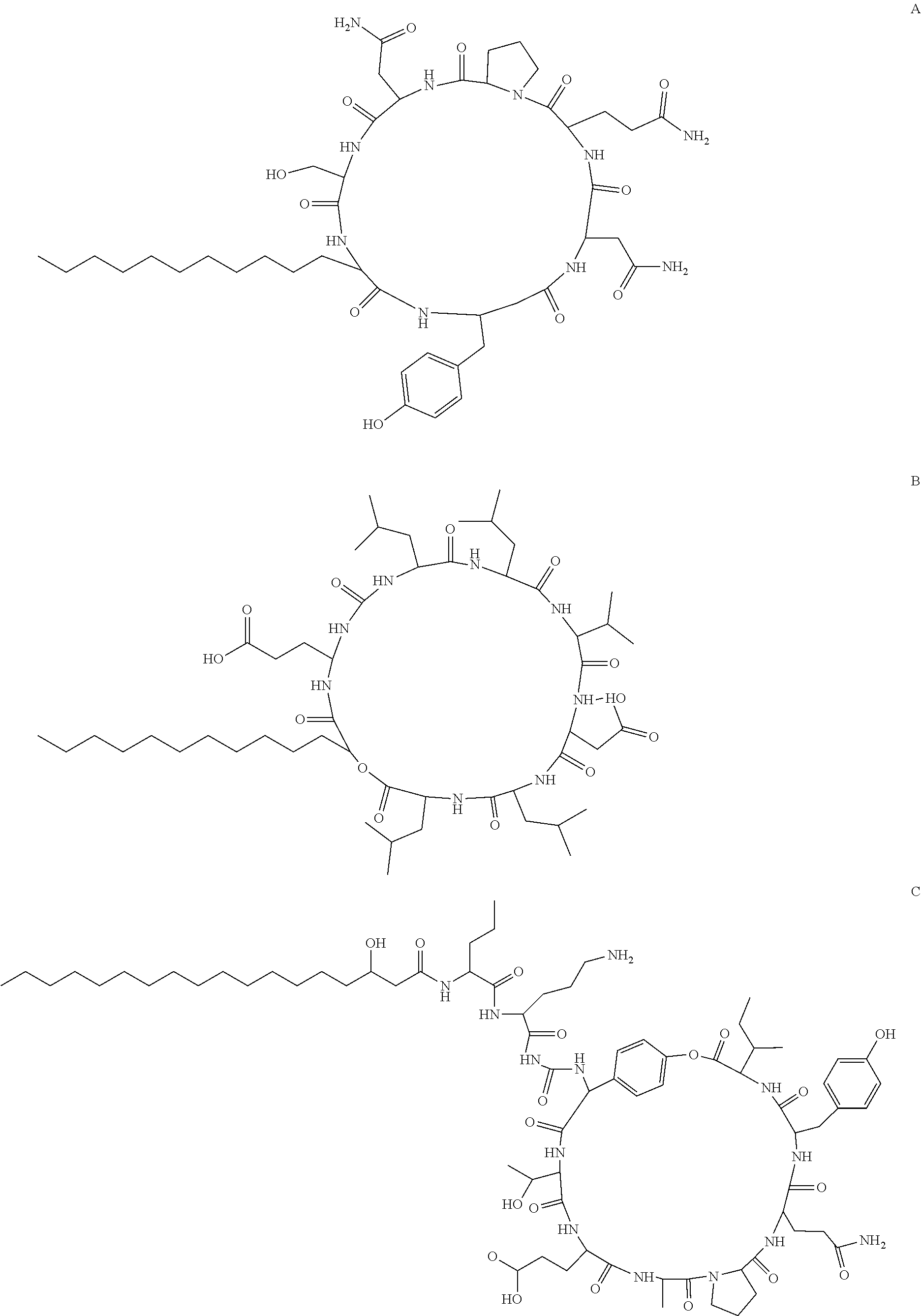 [SMILES: CCCCCCCCCCCC1NC(=O)C(CO)NC(=O)C(CC(N)=O)NC(=O)C2CCCN2C(=O)C(CCC(N)=O)NC(=O)C(CC(N)=O)NC(=O)CC(Cc2ccc(O)cc2)NC1=O ; CCCCCCCCCCCC1OC(=O)C(CC(C)C)NC(=O)C(CC(C)C)NC(=O)C2CC(=O)ON2C(=O)C(C(C)C)NC(=O)C(CC(C)C)NC(=O)C(CC(C)C)NC(=O)NC(CCC(=O)O)NC1=O ; CCCCCCCCCCCCCCCC(O)CC(=O)NC(CCC)C(=O)NC(CCCN)C(=O)NC(=O)NC1C(=O)NC(C(C)O)C(=O)NC(CCC(O)O)C(=O)NC(C)C(=O)N2CCCC2C(=O)NC(CCC(N)=O)C(=O)NC(Cc2ccc(O)cc2)C(=O)NC(C(C)CC)C(=O)Oc2ccc1cc2]

### Example 8—Surfactant HLB Values Based on Intended Property

Commercial surfactant-based products are used in food manufacturing, pharmaceuticals, cosmetics, personal care products, detergents, paints, textiles, fuels, natural and synthetic oils, and many other applications. In agriculture, they can be used as pesticides and/or fertilizers. They can also be used for ore enrichment, remediation of xenobiotics, and in oil and gas recovery.

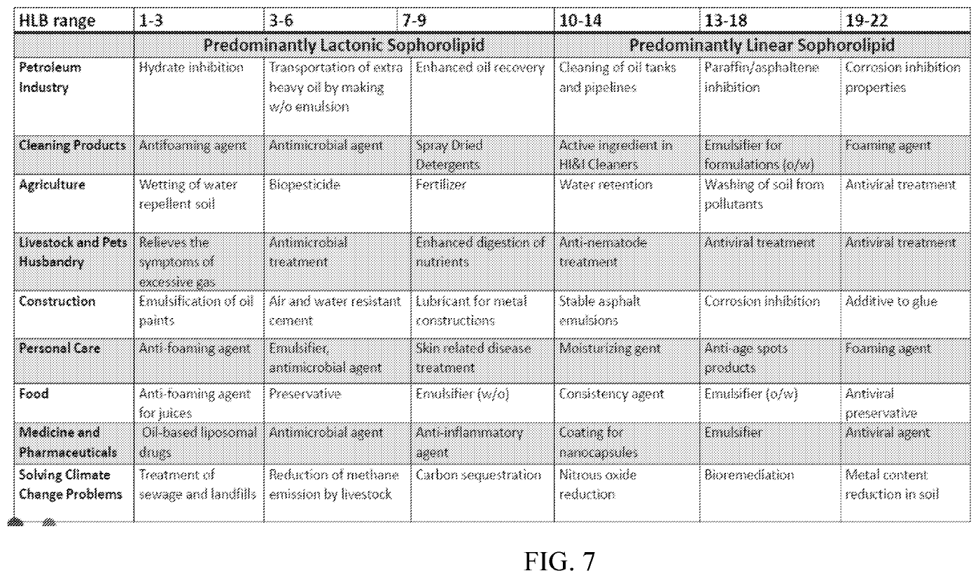

The choice of surfactant(s) depends upon the specific intended use and is determined based on the HLB value(s). Table 4 shows exemplary HLB values based on the desired property (see also FIG. 7).

TABLE 4
Surfactant HLB value based on desired property.

| HLB Value | Desired Properties |
|---|---|
| 1-3 | Anti-foaming |
| | Emulsification of oils with unlike properties |
| | (e.g., saturated and non-saturated oils) |
| 3-6 | W/O emulsification |
| | O/W demulsification |
| | Antimicrobial |
| 7-9 | Wetting alteration |
| | Increased wettability of powders in oil |
| | Self-emulsification of oils |
| 10-14 | O/W emulsification |
| | Detergent/cleansing |
| | Solubilization |
| 13-18 | O/W emulsification |
| | W/O demulsification |
| | Dispersant |
| | Solubilizing O/W emulsions (micro-emulsion |
| | formation) |
| 19-22 | O/W emulsification |
| | W/O demulsification |
| | Foaming |

### Example 9—Surfactant HLB Values Based on Intended Use—Petroleum Industry

Surfactants are widely used in oil and gas recovery, including, for example, in enhancement of crude oil recovery; stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 5 below for oil and gas recovery:

TABLE 5
HLB value based on desired application for petroleum industry.

| HLB | |
|---|---|
| Value | Uses |
| 1-3 | Prevention of hydrate formation and destruction of hydrates |
| | Super-xylene production due to high level of non-polarity |
| 3-6 | Transportation of extra heavy oil (W/O emulsification) |
| | Prevention of hydrate formation and destruction of hydrates |
| | (W/O emulsions prevent water from contact with gas) |
| | Biocide against sulfate-reducing bacteria (SRB) and reduction |
| | in H2S/prevention of H2S production |
| | Enhanced performance for oil-based drilling fluids and oil- |
| | based pump friction reducers |
| 7-9 | Conversion of petcoke powder to liquefied fuels |
| | Tar sands treatment for increased oil recovery |
| | Modification of reservoir wettability |
| | Enhanced oil recovery and well stimulation |
| | Enhanced proppant performance in fracking |
| 10-14 | Cleaning of tanks and pipelines |
| | Oil waste treatments and bioremediation for water and ground |
| | surfaces |
| | Reduction of heavy oil viscosity |
| | Enhanced performance for water-based drilling fluids |
| 13-18 | Demulsification of W/O emulsions |
| | Prevention of paraffin/asphaltene accumulation |
| | Paraffin wax dispersion |
| | Cleaning of downstream equipment and flow-lines |
| | Corrosion inhibition and prolonging lifetime for equipment |
| | and pipelines |
| 19-22 | Demulsification of W/O emulsions |
| | Corrosion inhibition and prolonging lifetime for equipment |
| | and pipelines |
| | Enhanced of foam and steam flooding |

In a specific exemplary embodiment, micelle size is another advantageous aspect for using biosurfactants in the oil and gas industry. Chemical surfactants and nanoparticle-containing fluids are often used for enhancing the recovery of oil from pores and hydraulic fractures in a formation. These compounds can range in size from 15-18 nm, up to about 100 nm. In certain formations containing, for example, shale, formation pore sizes are in the low-nanometer range, often from 13 to 18 nm; thus, utilizing biosurfactants according to the subject invention having, for example, less than 1.5 nm in size, provides a means of reaching the smallest pores to mobilize oil that other treatments cannot.

Accordingly, methods are provided for recovering oil from an oil-bearing formation having pore sizes less than 20 nm, less than 18 nm, less than 15 nm, and/or less than 13 nm, wherein a well treatment fluid comprising biosurfactants produced according to the subject invention is introduced into the formation, and wherein the treatment fluid contacts the oil present in the pores and mobilizes the oil therefrom, such that the oil is recovered from the formation in an amount that is greater than if a chemical surfactant was used in the well treatment fluid.

### Example 10—Surfactant HLB Values Based on Intended Use—Agriculture Industry

Surfactants are widely used in the agriculture industry. The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 6 below for agriculture:

TABLE 6
HLB value based on desired application for agriculture industry.

| HLB | |
|---|---|
| Value | Uses |
| 1-3 | Wetting of hydrophobic soils |
| 3-6 | Antibacterial and antifungal pesticide |
| | Co-surfactant for herbicides |
| 7-9 | Enhanced adsorption of water-insoluble nutrients |
| | Treatment of saline soil to reduce salinity (HLB 8-11) |
| | Enhanced water retention in soil |
| | Protection from soil erosion |
| | Spray drift reduction (for field treatments) |
| 10-14 | Washing pollutants from soil |
| | Enhanced adsorption of water-insoluble nutrients |
| | Treatment of saline soil to reduce salinity (HLB 8-11) |
| | Enhanced water retention in soil |
| | Protection from soil erosion |
| 13-18 | Washing pollutants from soil |
| | Antiviral pesticide |
| | Pesticide adjuvant/delivery systems |
| 19-22 | Enhanced water retention for dry soils |
| | Antiviral pesticide |

In a specific exemplary embodiment, micelle size is another advantageous aspect for using biosurfactants in the agriculture industry. In some instances, the small micelle size allows for penetration and uptake of the biosurfactant, as well as water and solubilized nutrients, into plant roots and vascular systems, allowing for reduced surface tension within the plant, increased nutrient and water transport into the plant cells, and increased excretion of toxins and waste matter out of the cells. Thus, plant health and growth can be increased as a result.

### Example 11—Surfactant HLB Values Based on Intended Use—Cosmetics and Personal Care

Surfactants are widely used in the cosmetics and personal care products industry. The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 7 below for cosmetics and personal care:

TABLE 7
HLB value based on desired application for
cosmetics and personal care products.

| HLB | |
|---|---|
| Value | Uses |
| 3-6 | Hydrophilic cleansing oils |
| | Topical products for treating burns, scars, wrinkles and/or acne |
| | Oral health products, such as toothpaste and mouth wash |
| | Antifungal treatments, such as seborrheic dermatitis and/or |
| | athlete's foot treatments |
| | Deodorant |
| | Perfume |
| | Hair dye |
| | Sunscreen |
| | Make-up, such as mascara, cream foundation, eye-liner, lipstick, |
| | lip balm, and/or lip gloss |
| | Lipo-gel |
| | Anhydrous products |
| | Waterproof formulations |
| 7-9 | Topical products for moisturizing skin, treating eczema, treating |
| | psoriasis and/or reducing cellulite |
| 10-14 | Hair and skin cleansers |
| | Topical products for reducing and/or preventing age spots |

### Example 12—Surfactant HLB Values Based on Intended Use—Cleaning Products

Surfactants are widely used in household, institutional and industrial (HI&I) cleaning products. The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 8 below for HI&I cleaning products:

TABLE 8
HLB value based on desired application
for HI&I cleaning products.

| HLB | |
|---|---|
| Value | Uses |
| 1-3 | Antifoaming agent in detergents where foam is unfavorable |
| 3-6 | Antimicrobial/antifungal soaps (without need for antibiotic |
| | or antifungal drugs) |
| | Sanitizers for surface disinfection |
| | W/O emulsification |
| 7-9 | Cleaning of porous surfaces (HLB up to 9) |
| | Pine oil and d-limonene cleaners |
| | Vehicle cleaners |
| | Spray-dried detergents |
| 10-14 | Active ingredient in detergent (as opposed to an adjuvant) |
| | Degreasing booster |
| | Vehicle cleaners |
| | Laundry detergent |
| | Dish soap/detergent |
| 13-18 | Active ingredient in detergent (as opposed to an adjuvant) |
| | O/W emulsification |
| 19-22 | Foaming agent in detergents |

### Example 13—Surfactant HLB Values Based on Intended Use—Construction

Surfactants are widely used in construction. The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 9 below for construction:

TABLE 9
HLB value based on desired application for construction.

| HLB | |
|---|---|
| Value | Uses |
| 1-3 | Emulsification of oil paints with unlike properties |
| | Anti-foaming agents |
| 3-6 | Emulsion-based latex paint (HLB 4-10) |
| | Antibacterial and/or antifungal agent |
| | Cement with prolonged durability and resistance to |
| | water and air degradation |
| 7-9 | Emulsion-based latex paint (HLB 4-10) |
| | Paint stripper (if kB value is high enough) |
| | Lubricant for metal equipment and machinery |
| 10-14 | Maximum stability of asphalt emulsions (70% asphalt, |
| | 30% water) |
| | Wetting agent for clays used in manufacture of bricks |
| | and ceramics |
| | Co-emulsifier for silicone in polishes |
| 13-18 | Post-added latex stabilizers (HLB 16-19) |
| | Dispersant for color pigments when dying additives |
| | are used (HLB 16-19) |
| | Corrosion inhibition |
| | Coatings (HLB 16-18) |
| | Additive to glue (HLB 16-19) |
| 19-22 | Concretes with increased (water-like) flow |
| | Corrosion inhibition |
| | Additive to glue (HLB 16-19) |

### Example 14—Surfactant HLB Values Based on Intended Use—Livestock Health

Surfactants are widely used in animal health. The choice of surfactant(s) depends upon the specific intended use and is determined based on the HLB value(s). Below are exemplary HLB values based on the desired use. Advantageously, the subject methods provide for surface-active compositions that can be adjusted to perform all of the functions as shown in Table 10 below for livestock and other domesticated animal health:

TABLE 10
HLB value based on desired application for
livestock and domesticated animal health.

| HLB | |
|---|---|
| Value | Uses |
| 1-3 | Relieves the symptoms of excessive gas in the GI tract: |
| | bloating, burping, flatulence |
| 3-6 | Antibacterial |
| | Antifungal |
| | Cytokine promotion |
| 7-9 | Enhanced digestion of fats (HLB 8-12) |
| | Spray drift reduction agents (treatment facilities) |
| | Anti-nematodal (HLB 9-12) |
| 10-14 | Enhanced digestion of fats (HLB 8-12) |
| | Improved absorption of nutrients |
| | Anti-nematode (HLB 9-12) |
| 13-22 | Antiviral treatment (non-acetylated ASL) |

In a specific exemplary embodiment, micelle size is another advantageous aspect for using biosurfactants in the livestock industry. In some instances, the small micelle size allows for penetration and uptake of the biosurfactant, as well as water, solubilized nutrients, and pharmaceuticals, through intestinal epithelial cells, and increased excretion of toxins and waste matter out of cells. In some instances, the small micelle size is also beneficial for penetrating and disrupting biofilms on surfaces, includes those of the GI tract, which can be helpful for reducing enteric methanogenic biofilms as well as other pathogenic biofilms.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

We claim:

1. A method for producing a green surfactant composition having one or more desired functional properties, the method comprising identifying a biosurfactant molecule having a specific functional property, and producing the biosurfactant molecule by cultivating a biosurfactant-producing microorganism,
wherein the functional properties of the composition and/or the biosurfactant molecule are measured by HLB value, CMC value, and/or KB value.

2. The method of claim 1, further comprising mixing the biosurfactant molecule with one or more additional biosurfactant molecules having specific functional properties at a ratio that will produce the one or more functional properties desired for the green surfactant composition.

3. The method of claim 1, wherein the structure of the biosurfactant molecule is modified during production of the biosurfactant by altering a parameter of fermentation while cultivating the biosurfactant-producing microorganism.

4. The method of claim 3, wherein the structure of the biosurfactant molecule is modified after production of the biosurfactant.

5. The method of claim 1, wherein the biosurfactant molecule is a glycolipid or a lipopeptide.

6. The method of claim 1, wherein the biosurfactant-producing microorganism is Bacillus amyloliquefaciens NRRL B-67928.

7. The method of claim 6, wherein the B. amyloliquefaciens NRRL B-67928 produces one or more lipopeptides selected from the group consisting of surfactin, lichenysin, fengycin and iturin.

8. A green surfactant composition having one or more desired functional properties, the composition comprising one or more biosurfactant molecules,
wherein the identity, ratio and structure of the one or more biosurfactant molecules are chosen based on their contribution to the desired functional properties.

9. The composition of claim 8, wherein the one or more biosurfactant molecules are glycolipids selected from the group consisting of sophorolipids, rhamnolipids, trehalose lipids, and mannosylerythritol lipids.

10. The composition of claim 8, wherein the one or more biosurfactant molecules are lipopeptides selected from the group consisting of surfactin, lichenysin, fengycin and iturin.

11. The composition of claim 8, wherein the one or more biosurfactant molecules are produced by Bacillus amyloliquefaciens NRRL B-67928.

12. The composition of claim 8, wherein the one or more biosurfactant molecules are produced by Starmerella bombicola.

13. The composition of claim 8, wherein the one or more biosurfactant molecules are produced by Wickerhamomyces anomalus.

14. The composition of claim 8, used for replacing and/or reducing chemical surfactants.

15. The composition of claim 8, wherein the biosurfactant has a micelle size of less than 5 nm.

16. A method of recovering oil from an oil-bearing formation having pore sizes less than 20 nm, wherein a well treatment fluid comprising a composition according to claim 5 is introduced into the formation, and wherein the treatment fluid contacts the oil present in the less than 20 nm pores and mobilizes the oil therefrom, such that the oil is recovered from the formation in an amount that is greater than if a chemical surfactant was used in the well treatment fluid.
